(19) United States
(12) Patent Application Publication
(10) Pub. No.: US 2020/0191081 A1
(43) Pub. Date: Jun. 18, 2020

(54) AUTOMOTIVE CARBON MONOXIDE DETECTOR WITH ABILITY TO ALERT DRIVER OR TURN OFF IGNITION

(71) Applicant: PANASONIC AUTOMOTIVE SYSTEMS COMPANY OF AMERICA, DIVISION OF PANASONIC CORPORATION OF NORTH AMERICA, Peachtree City, GA (US)

(72) Inventors: William Hughes, Commerce Township, MI (US); Rohan Repale, Farmington Hills, MI (US)

(73) Assignee: Panasonic Automotive Systems Company of America, Division of Panasonic Corporation of North America

(21) Appl. No.: 16/708,927

(22) Filed: Dec. 10, 2019

Related U.S. Application Data
(60) Provisional application No. 62/779,616, filed on Dec. 14, 2018.

(51) Int. Cl. F02D 41/04; F02D 41/02; F02N 11/08; F02D 41/22; F02N 11/10; B60Q 9/00; G01N 33/00

(52) U.S. Cl. CPC F02D 41/042; F02D 41/0215; F02N 11/087; F02D 41/22; F02N 2200/0802; B60Q 9/00; G01N 33/004; F02D 2200/50; F02D 2041/228; F02N 11/10

(57) ABSTRACT

A driver alert arrangement for a motor vehicle includes a sensor detecting a level of carbon monoxide associated with the motor vehicle and transmitting a signal indicative of the detected level of carbon monoxide. An engine ignition has an ON state in which the engine ignition enables operation of an engine of the motor vehicle, and an OFF state in which the engine ignition disables operation of the engine of the motor vehicle. An electronic processor is communicatively coupled to the sensor and to the engine ignition. The electronic processor receives the signal from the sensor and controls whether the engine ignition is in the ON state or the OFF state dependent upon the signal.

### CROSS-REFERENCED TO RELATED APPLICATIONS

This application claims benefit of U.S. Provisional Application No. 62/779,616 filed on Dec. 14, 2018, the disclosure of which is hereby incorporated by reference in its entirety for all purposes.

### BACKGROUND OF THE INVENTION

### 1. Field of the Invention

The present invention relates to a method of enhancing the safety of humans and animals sharing an enclosed space with a motor vehicle.

### 2. Description of the Related Art

There are incidents of humans/pets being subjected to dangerously high levels of carbon monoxide exhaust gas while inside or outside a motor vehicle. As the automotive technology advances, modern-day vehicles have become quieter. When vehicles use a push-button ignition, there is hardly a need to use the key fob. However, with new technology, there can be new unforeseen problems. One of these problems was reported in news when a couple died of exhaust fumes from their car being left running in their garage. The couple had their bedroom above the garage and the fumes seem to have permeated through the ceiling into the house.

The car exhaust fumes are predominantly composed of carbon monoxide, a colorless, odorless gas that is highly lethal to humans. Therefore, it is not advisable to let a vehicle idle in an enclosed space. However, mistakes do happen.

### SUMMARY OF THE INVENTION

The invention may provide a carbon monoxide sensor on the exterior of a motor vehicle. The sensor may be capable of operating with minimal power even when the vehicle is not running. The sensor may read the carbon monoxide levels in the parking space around the vehicle and take necessary steps to address the problem.

In order to address increasing carbon monoxide levels in the vicinity of the vehicle, the invention may provide in-vehicle notification using the head unit, instrument cluster, chimes etc.; provide cellphone notification if the vehicle is unoccupied; turn off the vehicle if it is idling with no one inside; and/or set off an alarm with notification. As an added feature, an interior carbon monoxide sensor can be used since the gas may permeate inside the vehicle and cause light headedness and eventual death.

The invention comprises, in one form thereof, a driver alert arrangement for a motor vehicle, including a sensor detecting a level of carbon monoxide associated with the motor vehicle and transmitting a signal indicative of the detected level of carbon monoxide. An engine ignition has an ON state in which the engine ignition enables operation of an engine of the motor vehicle, and an OFF state in which the engine ignition disables operation of the engine of the motor vehicle. An electronic processor is communicatively coupled to the sensor and to the engine ignition. The electronic processor receives the signal from the sensor and controls whether the engine ignition is in the ON state or the OFF state dependent upon the signal.

The invention comprises, in another form thereof, a driver alert method for a motor vehicle, including detecting a level of carbon monoxide associated with the motor vehicle. A signal indicative of the detected level of carbon monoxide is transmitted. An engine ignition is provided having an ON state in which the engine ignition enables operation of an engine of the motor vehicle, and an OFF state in which the engine ignition disables operation of the engine of the motor vehicle. The signal is received from the sensor. Whether the engine ignition is in the ON state or the OFF state dependent upon the signal.

The invention comprises, in yet another form thereof, a driver alert method for a motor vehicle, including detecting that a level of carbon monoxide outside the motor vehicle is above a first threshold level. A human driver of the motor vehicle is alerted about the detected level of carbon monoxide outside the motor vehicle being above the first threshold level. A level of carbon monoxide inside the motor vehicle is determined. If the determined level of carbon monoxide inside the motor vehicle is below a second threshold level, the driver is prompted to close a window of the motor vehicle. If the determined level of carbon monoxide inside the motor vehicle is instead above the second threshold level, the driver is prompted to open the window of the motor vehicle.

An advantage of the present invention is that it may avoid fatal consequences in the event of a vehicle being left running in an enclosed space.

Another advantage of the invention is that it may provide a notification if carbon monoxide is at excessively high levels in the vicinity of the vehicle, such as in a garage. Thus, the invention may avoid the dangerous gas permeating into the house or car and causing fatal accidents.

Yet another advantage of the invention is that it may prevent people from intentionally harming themselves by exposing themselves to poisonous fumes.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 is a block diagram of one embodiment of an automotive carbon monoxide detection arrangement of the present invention.

FIG. 2 is a flow chart of one embodiment of an automotive carbon monoxide detection method of the present invention.

FIG. 3 is a flow chart of one embodiment of a driver alert method of the present invention for a motor vehicle.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

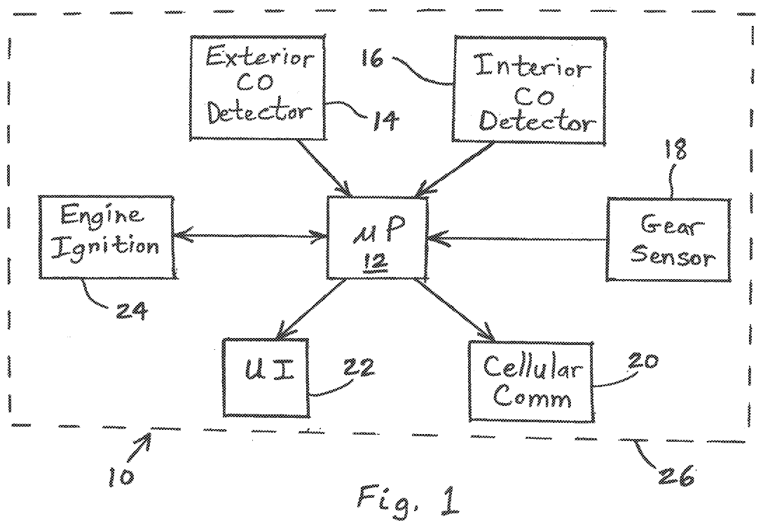

FIG. 1 illustrates one embodiment of an automotive carbon monoxide detection arrangement 10 of the present invention including an electronic processor 12, an exterior carbon monoxide detector 14, an interior carbon monoxide detector 16, a gear sensor 18, a wireless (e.g., cellular) communication module 20, a user interface 22 and an engine ignition 24. All of the above-described components of arrangement 10 may be disposed in or on a single motor vehicle 26.

Exterior carbon monoxide detector 14 may be disposed on an exterior surface of vehicle 26, and may measure a concentration or level of carbon monoxide outside of vehicle 26. Interior carbon monoxide detector 16 may be disposed within vehicle 26, and may measure a concentration or level of carbon monoxide inside a passenger compartment of vehicle 26. A gear sensor 18 may detect which of the gears (e.g., park, reverse, neutral, drive, low) that vehicle 26 is in. Wireless communication module 20 may enable wireless communication with a personal electronic device (e.g., a cell phone) of a user of vehicle 26, such as via a text message. A user interface 22 may include a display screen, head up display, and/or a loudspeaker disposed within the passenger compartment of vehicle 26. Engine ignition 24 may control whether an engine of vehicle 26 is running or not.

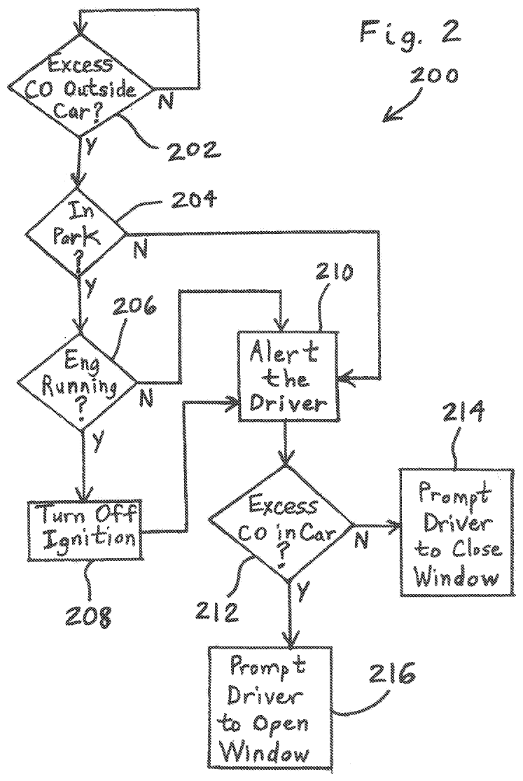

FIG. 2 illustrates one embodiment of an automotive carbon monoxide detection method 200 of the present invention. In a first step 202, it is determined whether there is an excessive level of carbon monoxide outside a motor vehicle. For example, processor 12 may receive signals from exterior CO detector 14, and, based on these signals, processor 12 may ascertain whether the carbon monoxide level outside of vehicle 26 exceeds a threshold level and is thus unsafe. If in step 202 it is determined that there is not an excessive level of carbon monoxide outside a motor vehicle, then operation remains at step 202 where the level of carbon monoxide continues to be monitored. On the other hand, if it is determined in step 202 that there is an excessive level of carbon monoxide outside a motor vehicle, then operation proceeds to step 204 where it is determined whether the vehicle is in the Park gear. For example, gear sensor 18 may transmit a signal to processor 12 indicating what gear the vehicle is in.

If it is determined in step 204 that the vehicle is in the Park gear, then operation proceeds to step 206 where it is determined whether the engine of the vehicle is running. For example, engine ignition 24 may transmit a signal to processor 12 indicating whether the engine ignition is turned ON, which would indicate that the engine of the vehicle is running.

If it is determined in step 206 that the vehicle engine is running, then operation proceeds to step 208 where the engine ignition is turned off. For example, processor 12 may turn off engine ignition 24. Operation then proceeds to step 210 where the driver is alerted that the level of carbon monoxide outside of the vehicle is excessively high. The driver may be alerted, for example, via a chime through a loudspeaker of user interface 22; via a visible indication produced by a head up display of user interface 22; or via a text message sent via cellular communication module 20 to the user's cell phone.

However, if it is determined in step 204 that the vehicle is not in the Park gear, then operation proceeds directly to step 210 where the driver is alerted that the level of carbon monoxide outside of the vehicle is excessively high. Similarly, if it is determined in step 206 that the vehicle engine is not running, then operation proceeds directly to step 210.

After the driver has been alerted in step 210, operation proceeds to step 212 where it is determined whether there is an excessive level of carbon monoxide inside the motor vehicle. For example, processor 12 may receive signals from interior CO detector 16, and, based on these signals, processor 12 may ascertain whether the carbon monoxide level inside of vehicle 26 exceeds a threshold level and is thus unsafe. If in step 212 it is determined that there is not an excessive level of carbon monoxide inside the motor vehicle, then operation proceeds to step 214 where the driver is prompted to close the window of the vehicle. For example, the driver may be prompted to close the window via an audible spoken message on a loudspeaker of user interface 22, or via a message on a display screen or a head up display of user interface 22.

On the other hand, if it is determined in step 212 that there is an excessive level of carbon monoxide inside the motor vehicle, then operation proceeds to step 216 where the driver is prompted to open the window of the vehicle. For example, the driver may be prompted to open the window via an audible spoken message on a loudspeaker of user interface 22, or via a message on a display screen or a head up display of user interface 22.

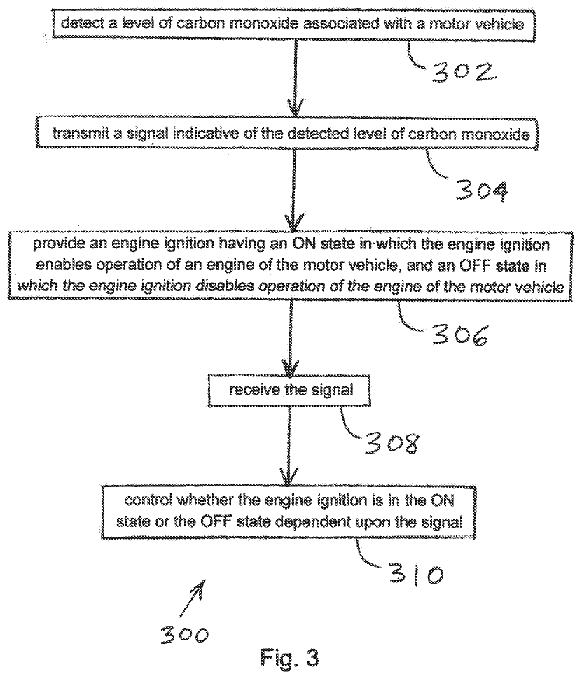

FIG. 3 illustrates one embodiment of a driver alert method 300 of the present invention for a motor vehicle. In a first step 302, a level of carbon monoxide associated with the motor vehicle is detected. For example, exterior carbon monoxide detector 14 may measure a concentration or level of carbon monoxide outside of vehicle 26.

Next, in step 304, a signal indicative of the detected level of carbon monoxide is transmitted. For example, exterior CO detector 14 may transmit a signal indicative of the carbon monoxide level outside of vehicle 26.

In a next step 306, an engine ignition is provided having an ON state in which the engine ignition enables operation of an engine of the motor vehicle, and an OFF state in which the engine ignition disables operation of the engine of the motor vehicle. For example, engine ignition 24 may control whether an engine of vehicle 26 is running or not. Engine ignition 24 has an ON state in which engine ignition 24 enables operation of an engine of motor vehicle 26, and an OFF state in which engine ignition 24 disables operation of the engine of motor vehicle 26.

In step 308, the signal is received. For example, processor 12 may receive the signals from exterior CO detector 14.

In a final step 310, whether the engine ignition is in the ON state or the OFF state is controlled dependent upon the signal. For example, processor 12 may turn off engine ignition 24 if the signal indicates that the level of carbon monoxide is high. Conversely, processor 12 may leave engine ignition 24 on if the signal indicates that the level of carbon monoxide is low.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A driver alert arrangement for a motor vehicle, comprising:
a sensor configured to detect a level of carbon monoxide associated with the motor vehicle and to transmit a signal indicative of the detected level of carbon monoxide;
an engine ignition having an ON state in which the engine ignition enables operation of an engine of the motor vehicle, and an OFF state in which the engine ignition disables operation of the engine of the motor vehicle; and
an electronic processor communicatively coupled to the sensor and to the engine ignition, the electronic processor being configured to:
receive the signal from the sensor; and
control whether the engine ignition is in the ON state or the OFF state dependent upon the signal.

2. The arrangement of claim 1, wherein the sensor is configured to detect a level of carbon monoxide outside of the motor vehicle.

3. The arrangement of claim 2, wherein the sensor comprises a first sensor, the arrangement further comprising a user interface and a second sensor configured to detect a level of carbon monoxide inside the motor vehicle, the processor being configured to use the user interface to prompt a human driver of the vehicle to close a window of the vehicle in response to the second sensor detecting that the level of carbon monoxide inside of the motor vehicle is below a threshold level.

4. The arrangement of claim 3, wherein the processor is configured to use the user interface to prompt the human driver to open a window of the vehicle in response to the second sensor detecting that the level of carbon monoxide inside of the motor vehicle is above the threshold level.

5. The arrangement of claim 1, further comprising a user interface, the processor being configured to use the user interface to alert a human driver of the vehicle that the level of carbon monoxide associated with the motor vehicle is above a threshold level.

6. The arrangement of claim 1, further comprising a gear sensor configured to detect a gear that the motor vehicle is in, the gear sensor being communicatively coupled to the processor, the processor being configured to control whether the engine ignition is in the ON state or the OFF state dependent upon the signal and dependent upon the gear that the motor vehicle is in.

7. The arrangement of claim 6, wherein the processor is configured to turn the engine ignition to the OFF state only if the motor vehicle is in a Park gear.

8. A driver alert method for a motor vehicle, comprising:
detecting a level of carbon monoxide associated with the motor vehicle;
transmitting a signal indicative of the detected level of carbon monoxide;
providing an engine ignition having an ON state in which the engine ignition enables operation of an engine of the motor vehicle, and an OFF state in which the engine ignition disables operation of the engine of the motor vehicle;
receiving the signal; and
controlling whether the engine ignition is in the ON state or the OFF state dependent upon the signal.

9. The method of claim 8, wherein the detecting step comprises detecting a level of carbon monoxide outside of the motor vehicle.

10. The method of claim 9, further comprising:
detecting a level of carbon monoxide inside the motor vehicle; and
prompting a human driver of the vehicle to close a window of the vehicle in response to detecting that the level of carbon monoxide inside of the motor vehicle is below a threshold level.

11. The method of claim 10, further comprising prompting the human driver to open a window of the vehicle in response to detecting that the level of carbon monoxide inside of the motor vehicle is above the threshold level.

12. The method of claim 8, further comprising alerting a human driver of the vehicle that the level of carbon monoxide associated with the motor vehicle is above a threshold level.

13. The method of claim 8, further comprising detecting a gear that the motor vehicle is in, the controlling step including controlling whether the engine ignition is in the ON state or the OFF state dependent upon the signal and dependent upon the gear that the motor vehicle is in.

14. The method of claim 13, wherein the controlling step includes turning the engine ignition to the OFF state only if the motor vehicle is in a Park gear.

15. A driver alert method for a motor vehicle, comprising:
detecting a level of carbon monoxide outside the motor vehicle, the detected level of carbon monoxide outside the motor vehicle being above a first threshold level;
alerting a human driver of the motor vehicle about the detected level of carbon monoxide outside the motor vehicle being above the first threshold level;
determining a level of carbon monoxide inside the motor vehicle;
in response to the determined level of carbon monoxide inside the motor vehicle being below a second threshold level, prompting the driver to close a window of the motor vehicle; and
in response to the determined level of carbon monoxide inside the motor vehicle being above the second threshold level, prompting the driver to open the window of the motor vehicle.

16. The method of claim 15 further comprising turning OFF an ignition of an engine of the motor vehicle in response to ascertaining that the motor vehicle is in a Park gear and that the engine is running.

17. The method of claim 15 wherein the alerting step includes alerting the driver via cellular communication from the motor vehicle to a personal electronic device of the driver.

18. The method of claim 17 wherein the alerting step includes alerting the driver via a text message.

19. The method of claim 15 wherein the alerting step includes alerting the driver via a head up display.

20. The method of claim 15 wherein the alerting step includes alerting the driver via an audible chime played on a loudspeaker of the motor vehicle.
